(19) United States
(12) Patent Application Publication
(10) Pub. No.: US 2004/0047921 A1
(43) Pub. Date: Mar. 11, 2004

(54) Liquid compositions comprising non-digestible oligosaccharides and green tea catechins, method and uses thereof

(76) Inventors: Donald L. Simmons, Dollard Des Ormeaux (CA); Cunji Dong, St-Laurent (CA)

Correspondence Address: MYERS BIGEL SIBLEY & SAJOVEC, PO BOX 37428, RALEIGH, NC, 27627, US

(21) Appl. No.: 10/601,241

(22) Filed: Jun. 20, 2003

Related U.S. Application Data
(60) Provisional application No. 60/390,150, filed on Jun. 21, 2002.

(51) Int. Cl. A61K 31/715; A61K 35/78

(52) U.S. Cl. 424/729; 514/54

(57) ABSTRACT

A method and liquid compositions for restoring and/or maintaining colon functionality and/or for helping to prevent cancer such as colon cancer. The method consisting in administering to a human being a liquid composition including an effective amount of a non-digestible oligosaccharide, at least one green tea catechin and a buffering agent mixture, said liquid composition being in a pH range of from about 4.7 to about 5.0. A method for making the liquid compositions is also disclosed.

### RELATED APPLICATION

This application claims priority from U.S. Provisional Patent Application No. 60/390,150, filed on Jun. 21, 2002, the disclosure of which is incorporated by reference herein in its entirety.

### BACKGROUND OF THE INVENTION

A) Field of the Invention

This invention relates to liquid compositions comprising a combination of non-digestible oligosaccharides (NDO) and the green tea catechin, epigallocatechin gallate (EGCG) and methods of making thereof. The invention also relates to the uses of such compositions for the restoration and the maintenance of colon health.

B) Brief Description of the Prior Art

The Functional Food Concept and the Healthy Gut

In recent years, the functional food concept has moved away from mineral and vitamin supplementation towards the situation where improved gut functionality is the main driving force. The key focus is a need to restore and maintain intestinal microbial balance in favor of friendly bacteria and to scavenge free radicals generated by metabolic processes. The colon is the most intensely populated and active microflora region (>10¹² cells per gram of dried contents) of the gastrointestinal tract and is therefore the main target for such dietary intervention.

Numerous external and internal factors such as stress, diet, drugs, aging and disease impact free radical production and upset the delicate microbial balance favoring friendly bifidobacteria and lactobacilli over pathogenic bacteria such as E. coli and Clostridium perfringens. Free radicals are believed to be responsible for the onset of carcinogenic transformation of normal cells. However, no single treatment exists to both scavenge free radicals in order to help prevent cancer and to help restore and maintain a friendly microflora for the gut.

Dietary Intervention and Modulation of the Human Gut Flora

Dietary modulation of the human gut flora has been carried out for years by the use of probiotics, i.e. live microbial feed supplements such as yogurts, fermented milks and kefirs, but the main drawbacks associated with these formulations are that they require refrigeration and have a short shelf life. A more recent approach (Gibson and Roberfroid, Journal of Nutrition, 1995, 125: pp. 1401-1412) is to increase numbers of friendly resident bifidobacteria and lactobacilli in the gut by ingestion of prebiotics. Prebiotics may be defined as non-digestible carbohydrates, e.g. oligosaccharides that pass through the small intestine undigested and are fermented in the colon.

Modulation of Gut Flora by Non-Digestible Oligosaccharides (NDOs)

Non-digestible oligosaccharides (NDOs) are described as soluble fibers and according to the International Unions of Pure and Applied Chemistry and Biochemistry (IUPAC-IUB) are defined as being composed of two to about ten monosaccharides linked together. However some longer chain saccharides such as inulin are included within this NDO definition but are more aptly defined as non-digestible; non-starch polysaccharides (NSP). Van Loo et al. (British Journal of Nutrition, 1999, 81:pp 121-132) conducted an extensive literature review and chemical analyses of non-digestible oligosaccharides that were allowed for human consumption in Europe, Asia and North America. This European Commission-funded project was established to perform a consensus exercise on the possible functional food properties of non-digestible oligosaccharides. The general consensus among participants was the existence of strong evidence for NDO increasing the numbers of friendly bacteria in the human large intestine as well as a favorable impact on bowel habit. Promising and preliminary evidence also exists that inulin or oligofructose ingestion may result in increased calcium absorption in humans, interact with functioning of lipid metabolism and a possible preventive effect against colon cancer from experimental animal studies.

Quantitative analyses of these NDOs were performed by special high-performance anion exchange chromatography followed by a post-column acid hydrolysis technique to convert the NDO into its constituent monosaccharides. The results demonstrated that only inulin and oligofructose were considered to be very pure, e.g., greater than 87 percent purity and that greater than 90 percent of their ingested amounts reached the colon. Both NDOs are allowed for human consumption in the United States along with soybean oligosaccharides. In the latter, NDO is a minor fraction, about 30 percent, and only about 50 percent of an ingested amount reaches the colon. Modulation of the gut flora by short-chain fructo-oligosaccharides (scFOS)

Of particular interest is a pure concentrated form (>94% purity) of short-chain oligofructoses or fructo-oligosaccharides (scFOS) produced commercially by a fermentation method. This scFOS (NutraFlora™, GTC Nutrition Co., Colorado) consists of 2, 3 and 4 fructose units linked linearly to a glucose unit and is thus considered to have a DP upper limit of 4 (where DP refers to “degree of polymerization” and is a measure of polymer size or chain length). Such scFOS are found in trace amounts in certain fruit, vegetables and grains notably, bananas, peaches, onions, artichokes, shallots, chicory root, garlic, rye and wheat.

In November 2000, NutraFlora™ received GRAS status (Generally Recognized as Safe) for use in foods in the United States. Over one hundred fifty scientific studies have shown known health benefits with the daily use of NutraFlora™ including: improved overall digestive function, improved immune function, improved absorption of calcium and magnesium, improved regularity and proliferation of indigenous beneficial intestinal microflora. Clinical studies reveal that as little as 1-gram NutraFlora™ taken daily increases significantly the bifidobacteria population in the colon.

Unlike their longer chain counterparts, the short-chain fructo-oligosaccharides, including oligofructose with a DP of 8 (Raftilose P95, Orafti Food Ingredients, Netherlands), are readily soluble (>75%) in water at room temperature. Conversely, inulin samples with average minimum DP values of 10 and 25 (Raftiline GR and Raftiline HP, respectively, Orafti Food Ingredients, Netherlands) possess water solubilities of only 12.5% and 2%, respectively. An additional feature of the shorter chain fructo-oligosaccharides is their more rapid fermentation and conversion by intestinal bacteria into short-chain fatty acids (SCFA). The latter lower colon pH, stimulate electrolyte absorption and create an unfavorable microenvironment for pathogenic bacteria.

Modulation of Gut Flora by Green Tea Catechins (GTC)

Green tea, derived from the plant Camellia Sinensis, contains polyphenols, which account for up to one third of its leaves dry weight. The major polyphenols are four catechins consisting of epicatechin (EC), epigallocatechin (EGC), epicatechin gallate (ECG), and epigallocatechin gallate (EGCG) with the latter being the most abundant. One commercial decaffeinated extract (Greenselect™, Indena, Italy) is available with an EGCG content exceeding 40 percent. The health benefits of tea polyphenols have been attributed to their antioxidant activity. EGCG is the key polyphenolic ingredient believed to be responsible for most of the cancer chemopreventive properties of green tea. Mukhtar et al. (Biochemistry Biophysics Research Communications, 2001, 288:1, 101-105) have demonstrated that EGCG inhibits topoisomerase I activity in human colon carcinoma cells (through a process called apoptosis or programmed cell death) but not in normal cells. Similar growth inhibition studies appear in the scientific literature for human leukemic cells by Otsuka et al. (Life Sciences, 1998, 63:16, 1397-1403) and for prostate cancer cell lines by Paschka et al. (Cancer Letters, 1998, 130:1-2,1-7). Research by Swiercz et al. (Oncology Reports, 1999, 6(3): 523-526) has shown EGCG to prevent cancer by binding to a proteolytic enzyme called urokinase, which is overexpressed in cancer. Because of EGCG, green tea catechins (GTC) are officially recognized in Japan as a cancer preventative. The National Cancer Institute in the USA is currently sponsoring studies to evaluate EGCG toxicity and its potential as a cancer chemopreventive agent (National Toxicology Program).

Chow et al. (Cancer Epidemiology Markers & Prevention, 2001, 10:1, 53-8) performed a pharmacokinetic study of tea polyphenols following single-dose administration of EGCG and a green tea extract (GTE) to human volunteers. Dose levels (200 to 800 mg) were administered on the basis of EGCG content. Blood and urine samples were collected over a 24-hour period and analyzed for catechin content by high-performance liquid chromatography (HPLC). Mean areas under the plasma concentration time-curves of free or unchanged EGCG were similar at all dose levels. Free EGC and EC were not detected in plasma after EGCG administration and were present at low/undetectable levels after GTE administration. However high concentrations of EGC and EC glucuronide/sulfate conjugates were found in plasma and urine after GTE administration. EGCG was not detected in the urine and the speculation was that elimination occurred in the bile.

Evidence for the fate of orally administered EGCG and GTE exists in two experiments conducted by Hara (Green Tea Health Benefits and Applications, by Y. Hara, Marcel Dekker 2001). In the first experiment involving rats, (Chapter 16, The Fate of Tea Catechins after Oral Ingestion), residual EGCG contents were monitored in the stomach, small intestine, large intestine and feces during a 20-hour period post EGCG administration. EGCG disappeared rapidly from the stomach into the small intestine and began to appear 2 hours later in the large intestine, reaching a maximum in this area 8 hours after ingestion and only appearing in the feces after 12 hours. Approximately 20% was unaccounted for during transit through the small intestine and assumed to be absorbed into the body.

In the second experiment (Chapter 15, Effects on Intestinal Flora) Hara demonstrated the non-digestibility and bifidogenic potential of green tea catechins (GTC) in a clinical study involving elderly residents in a long-term care facility. Daily administration of GTC (300 mg, 28% EGCG content) was conducted during a three-week interval with fecal specimens collected at the end of each week, as well as just before and one week after. Levels of lactobacilli and bifidobacteria increased significantly during the study with a concomitant decrease in levels of Enterobacteriaceae and Clostridium. There was also an overall increase in fecal mass and decrease in fecal ammonia, sulfide and other odorous metabolites.

Dietary Supplements Versus Functional Foods

As a dietary supplement, GTC is available almost exclusively in capsule or tablet formulations, usually in combination with other antioxidants, minerals and vitamins. Some GTC products may contain FOS but in insufficient quantities to provide any significant health benefit. For instance, U.S. Pat. No. 5,681,569 describes dry and fluid beverage compositions containing green tea solids, electrolytes and carbohydrates to provide improved cellular hydration and drinkability. Although the drinkable beverage comprises a maximum of 0.35% green tea flavanols (catechins), which is a minimum requirement for any significant health benefit, the preferable pH range of from about 2.5 to about 4.0 is in practice unacceptable from an FOS stability standpoint.

Fructo-oligosaccharides (FOS) are relatively stable in aqueous solution provided the pH is in the neutral range. Freitas et al. (Institute of Food Technologists 2001 meeting, Session 44E-25) observed a 42 to 64% FOS loss with a corresponding increase of the breakdown product fructose following pasteurization of carrot-orange juice samples spiked with FOS. The juice pH ranged between 3.55 and 3.92. The conclusion reached was that FOS hydrolysis into fructose occurred due to the high temperature associated with pasteurization and the acid pH effect. L'Homme et al. (Journal of Agriculture Food Chemistry, 2003, 51:pp 224-228) compared the hydrolytic degradation profiles of the individual components of scFOS (GF2, GF3 and GF4) in aqueous solutions buffered at 4.0, 7.0 and 9.0 and stored at five temperatures ranging between 80 and 120° C. The results demonstrated an improved stability with increasing chain length for pH 4.0 samples, superior stability profiles at pH 7.0 and no apparent degradation of samples stored at pH 9.0 during similar storage time intervals. Mitchell et al. (U.S. Pat. No. 5,422,346) recognized the influence of pH control in isolating a stable polymorphic form of inulin from dahlia tubers. In order to obtain an inulin polymer mixture having greater than 50% of the polymers in the range of DP 10 to DP 45, it was necessary to stabilize the pH to about 7 in order to limit acid degradation of the smaller inulin polymers.

U.S. Pat. No. 5,681,569, among others, teaches that an edible acid such as malic, citric, tartaric, fumaric and the like may be used in order to maintain a pH of less than 4.6. However, pH maintenance is never achieved, in practice, by an acid alone. Indeed, pH maintenance also requires careful attention be paid to buffering agents and buffer capacity. Although citric acid, among edible organic acids, and its sodium salt are capable of maintaining strong buffer capacity—the other acids taught in the prior art, namely US '569, are rarely if ever used for the purpose of maintaining pH in such beverage drinks. Maintaining pH control is a major problem, especially in flavored oral solutions containing high solids content.

Similarly, U.S. Pat. No. 4,946,701 describes pH preferences for carbonated beverages containing green tea solids or flavanols, also in the range of 2.5 to about 4.0, with the same inherent stability drawbacks and problems associated with FOS inclusion. One of the many problems with flavanols is their relatively poor stability in liquids hence the reason behind assigning only 3-months stability for such beverages stored at room temperature. Ekanayake et al. (U.S. Pat. No. 6,268,009) describe diet beverages containing a highly purified aqueous green tea extract (1% solids content containing a mixture of 4 catechins), along with theanine, minerals, caffeine, the sweetener aspartame and other sweeteners selected from fruit or vegetable juices. In such beverages, green tea extract inclusion is used merely in order to suppress the aftertaste associated with aspartame. Furthermore, in view of the fact that the maximum EGCG content is of 0.084%, no significant health benefit per se may be expected from such beverages.

Many canned and bottled tea drinks containing GTC are known and available in Asia. However, a common and inherent problem associated with these tea drinks is the relatively high amount of EGCG degradation product observed in these products. Indeed, a stability evaluation performed by Chen et al. (Journal of Agriculture Food Chemistry, 2001, 49: 477-482) showed that extensive degradation of EGCG occurred after only 30 minutes heating in water at 980 C and additional losses were minimal on continued heating to 7 hours. The degradation was also found to be pH dependent whereby a higher pH value led to an unstable environment for EGCG. In a similar pH versus temperature study, Proniuk et al. (Journal of Pharmaceutical Sciences, 2002, 91: 111-116) observed complete loss of EGCG at 50° C. of samples buffered with citric acid—sodium citrate at pH 5.0 after only 7 days. Far greater stability was observed at pH 3, with—40% EGCG content remaining after 28 days at 50° C. The effect of different oxygen concentrations in citrate buffered samples at pH 4, 5, 6 and 7 were analysed by Zimeri and Tong (Journal of Food Science, 1999, 64(5): 753-758) who demonstrated that more rapid degradation of EGCG occurred with increasing pH and oxygen concentration.

The results of the above-mentioned studies all point to the acutely felt, longstanding and as yet unresolved problem of extended shelf life at room temperature for such formulations. In view of the aforementioned reports stemming from both the scientific and patent literature, there thus exist real and inherent difficulties in attempting to formulate a stable liquid product containing both EGCG and non-digestible oligosaccharides (NDOs) in combination. The first of this series of problems, is the matter of NDO solubility itself. Indeed, only the short-chain fructo-oligosaccharides (scFOS) with DP≦4 or oligofructose (FOS) with DP≦8 possess the desired solubility profiles for liquid formulations. A second and additional concern is the maximum amount of GTE to be added to the formulation in light of the astringent nature associated with this EGCG source. Of far greater importance, is yet another problem which only compounds the first two difficulties: e.g. how to stabilize these two ingredients in combination EGCG being more stable in an acid medium while FOS is more comfortable in a neutral environment, in order to attain longer or extended shelf life. In view of all of the above, there is thus a need for liquid compositions comprising, in combination, non-digestible oligosaccharides, such as fructo-oligosaccharides (FOS), and the green tea catechin, epigallocatechin gallate (EGCG), for helping to prevent cancer, such as colon cancer, and for restoring and maintaining colon functionality.

There is also a need for liquid compositions comprising non-digestible oligosaccharides such as fructooligosaccharides (FOS) and the green tea catechin epigallocatechin gallate, in combination, which have an increased efficacy, stability and shelf life. There is also a need for a method for making such liquid compositions comprising non-digestible oligosaccharides such as fructooligosaccharides (FOS) and the green tea catechin epigallocatechin gallate, in combination, which have an increased efficacy, stability and shelf life.

The present invention fulfils these needs and also other needs which will be apparent to those skilled in the art upon reading the following specification.

### SUMMARY OF THE INVENTION

According to one aspect of the present invention, there is provided a liquid composition for providing restored or maintained colon functionality comprising an effective amount of a non-digestible oligosaccharide, at least one green tea catechin and a buffering agent mixture, said liquid composition being in a pH range of from about 4.7 to about 5.0.

According to an other aspect of the present invention, there is also provided a liquid composition for preventing cancer comprising an effective amount of a non-digestible oligosaccharide, at least one green tea catechin and a buffering agent mixture, said liquid composition being in a pH range of from about 4.7 to about 5.0.

According to a further aspect of the present invention, there is provided a method of restoring and maintaining colon functionality in a human being, said method comprising administering to said human being an effective amount of a liquid composition of the present invention to restore and maintain colon functionality in said human being.

According to an other aspect of the present invention, there is provided a method of helping prevent cancer in a human being, said method comprising administering to said human being an effective amount of a liquid composition of the present invention to help prevent cancer in said human being.

According to yet an other aspect of the present invention, there is also provided a use of an effective amount of a liquid composition of the present invention to restore and maintain colon functionality in a human being.

According to still an other aspect of the present invention, there is provided a use of an effective amount of a liquid composition of the present invention to help prevent cancer in a human being.

According to an other aspect of the present invention, there is provided a use of an effective amount of a liquid composition of the present invention for the manufacture of a medicament useful for restoring or maintaining colon functionality in a human being.

According to yet an other aspect of the present invention, there is provided a use of an effective amount of a liquid composition of the present invention for the manufacture of a medicament useful for in helping to prevent cancer in a human being.

According to still an other aspect of the present invention, there is provided a method for increasing the stability of short-chain fructo-oligosaccharides and epigallocatechin gallate (EGCG) in liquid compositions comprising the steps of:

buffering the said liquid with sodium citrate and citric acid in a pH range of from about 4.70 to about 5.0 and a minimum buffer capacity of 50 mM;

adding to the said liquid an oxygen scavenging agent to compete for removal of dissolved oxygen; and

adding a trace metal ion scavenger to remove trace metal ions.

### DETAILED DESCRIPTION OF THE INVENTION

Liquid Compositions with Non-Digestible Oligosaccharides and the Green Tea Catechin, Epigallocatechin Gallate

According to the present invention, there are provided liquid compositions for restoring and/or maintaining colon functionality and for helping prevent cancer comprising an effective amount of a non-digestible oligosaccharide, at least one green tea catechin, and a buffering agent mixture, the liquid composition being in a pH range of from about 4.7 to about 5.0. Liquid compositions are preferred because they have a far greater acceptability among children and the elderly and because they avoid the multiple daily dosing requirements for non-digestible oligosaccharide-containing solid dosage forms such as capsules and tablets.

Non-Digestible Oligosaccharides (NDOs)

Preferably the non-digestible oligosaccharide is comprised in the liquid composition of the present invention at a concentration of about 3% to about 45% (all weights given herein are by percentages unless otherwise specified). Preferably, the non-digestible oligosaccharide is chosen from xylo-oligosaccharides, soyoligosaccharides, fructo-oligosaccharides, trans-galactooligosaccharides, palatinose condensates, isomalto-oligosaccharides, inulin, pyrodextrin, and the like, and mixtures thereof. More preferably, the non-digestible oligosaccharide is water soluble and consists of oligofructose, and short chain fructo-oligosaccharides and mixtures thereof, and even more preferably, of short chain fructo-oligosaccharides.

Epigallocatechin Gallate (EGCG)

Of course EGCG may be comprised in the liquid composition of the present invention individually or in combination with other catechins. Preferably the green tea catechin, epigallocatechin gallate (EGCG), is present at a concentration of about 0.1 to about 0.8%, EGCG may be obtained by any suitable method known in the art. Even more preferably, EGCG is obtained from a fermented or unfermented green tea extract and the like. EGCG may also be obtained from a caffeine-containing or decaffeinated green tea extract and the like. Even more preferably still, the composition comprises from about 0.1 to about 0.8% of EGCG from a decaffeinated green tea plant extract having an EGCG content of from about 25 to 99 percent by weight. EGCG may also be obtained by synthesis.

Trace Metal Ion Scavenger, Antioxidant, and Buffering Agent Mixture

The liquid composition of the invention also comprises from about 0.05 to about 0.25% of an acceptable trace metal ion scavenger; from about 0.1% to about 5% of an acceptable oxygen scavenging agent; and from about 0.1% to about 2% of an acceptable buffering agent mixture.

Preferably, the trace metal ion scavenger for the liquid composition in the form of aqueous solution is selected from the group consisting ethylene diamine tetracetic acid (EDTA) and the like and salts thereof, and mixtures thereof. Preferably, the acceptable oxygen scavenging agent is selected from butylated hydroxytoluene (BHT), butylated hydroxyanisole (BHA), tocopherols, ascorbic acid and its salts, anthocyandins from fruit or vegetable juice powders or concentrates, and the like, and mixtures thereof. More preferably, it consists of ascorbic acid and anthocyandins from berry juice powders.

Preferably, the buffering agent mixture is selected from citrates, phosphates, acetates, ascorbates, and the like, and mixtures thereof. More preferably, it consists of a mixture comprising sodium citrate and citric acid.

Sweetening, Vicosity, Preservative

The liquid composition of the invention may further comprise from about 1% to about 30% of an acceptable sweetening agent; from about 0.1% to about 2% of an acceptable viscosity promoting agent; from about 0.1% to about 10% of a flavor agent; from about 0.1% to about 1% of an acceptable preservative;

Preferably, the sweetening agent is selected from glucose, fructose, sucrose, maltodextrin, corn syrups, xylitol, mannitol, sorbitol, stevioside, aspartame, sodium cyclamate, sodium saccharin, and the like, and mixtures thereof. More preferably, the sweetening agent is selected from xylitol and fructose, and mixtures thereof.

Preferably, the viscosity-promoting agent is selected from the group consisting of guar gum, xanthan gum, alginates, carboxymethylcelluose (CMC), gum arabic, carageenan, locust bean gum, tragacanth, agar agar, and the like, and mixtures thereof. More preferably, the viscosity-promoting agent is selected from carrageenan.

The flavoring agent may be derived from natural sources or it may be synthetically prepared. Preferably it is selected from fruit juice, fruit flavors, botanical flavors, and the like, and mixtures thereof.

Preferably, the preservative is selected from the gorup consisting of methylparaben, propylparaben, butylparaben, benzoic acid, sorbic acid, hexametaphosphates, and the like, and mixtures thereof. More preferably, it consists of methylparaben.

Accordingly, the liquid composition of the present invention may comprise:

a) from about 3 to about 45 percent of short-chain fructooligosaccharides;

b) from about 0.1 to about 0.8 percent of epigallocatechin gallate (EGCG);

c) from about 1 to about 20 percent of an acceptable sweetening agent;

d) from about 0.1 to about 2 percent of an acceptable viscosity promoting agent;

e) from about 0.1 to about 2 percent of an acceptable buffering agent mixture;

f) from about 0.1 to about 10 percent of a flavor agent;

g) from about 0.1 to about 1 percent of an acceptable preservative;

h) from about 0.1 to about 0.25 percent of an acceptable trace metal ion scavenging agent;

i) from about 0.1 to about 5 percent of an acceptable oxygen or free radical scavenging agent; and

j) from about 50 to about 80 percent water.

The compositions of the invention may be prepared by any conventional methods known in the art.

As discussed herein, the most advantageous composition is a liquid composition which has far greater acceptability over tablets and capsules among children and the elderly. An additional advantage of liquid compositions is the greater flexibility in dosage adjustment for active ingredient administration. Numerous flavors are available to overcome undesirable taste problems such as astringency associated with green tea extracts. In the following liquid composition examples, GTE concentrations were fixed to a maximum 0.35%, equivalent to approximately 18 mg EGCG per 15 mL, and then supplemented with pure EGCG to label claim in order To minimize flavoring difficulties. The following liquid composition examples are illustrative of the wide range of applicability of the present invention and are not intended to limit its scope. Modifications and variations can be made therein without departing from the spirit and scope of the invention. For example, a person skilled in the art can easily formulate variations of this invention in forms such as oral suspensions, beverages, powdered drink blends, conventional and chewable tablets, capsules and delayed release dosage forms. Although any method and material similar or equivalent to those described herein can be used in the practice for testing of the present invention, the preferred methods and materials are described.

Maintenance and Restoration of Colon Functionality and Cancer Prevention

The liquid compositions of the present invention are particularly useful to help restore and maintain a balance favoring friendly microflora in the colon of human beings and to simultaneously scavenge for free radicals generated by metabolic activities. Therefore, according to an other aspect of the invention, it is provided a method to restore and maintain colon functionality comprising administering a liquid composition of the present invention to a human being.

The liquid compositions of the present invention may also be used to help prevent cancer, and more particularly gastrointestinal-related cancers such as colon cancer. Therefore, according to yet an other aspect of the invention, it is provided a method to help prevent cancer, comprising administering a liquid composition of the present invention to a human being.

In such cases, the amount of non-digestible oligosaccharides and the green tea catechin, epigallocatechin gallate, present in the compositions of the present invention is a therapeutically effective amount. A therapeutically effective amount is that amount necessary for non-digestible oligosaccharides and the green tea catechin, epigallocatechin gallate, to perform their biological function in a synergistic manner (i.e. enhance or help maintain beneficial microflora in the colon, promote bowel regularity, scavenge for metabolic free radicals and to help prevent cancer) without causing human beings overly negative effects. The exact amount of these bioactive agents to be used and administered will vary according to the following factors: their activity and/or purity, the type of condition being treated, the age and/or weight of the individual, the mode of administration, as well as the other ingredients in the composition.

Method to Increase Stability of Short-Chain Fructo-Oligosaccharides (scFOS) and the Green Tea Catechin, Epigallocatechin Gallate (EGCG) in Liquid Compositions.

According to yet an other aspect of the invention, it is provided a method to increase the stability of short-chain fructo-oligosaccharides (FOS) and the green tea catechin, epigallocatechin gallate (EGCG) in liquid compositions. According to a preferred embodiment, the method comprises the steps of: buffering a liquid solution with sodium citrate and citric acid at a pH range of from about 4.7 to about 5,0, and more preferably at pH 4.80, and a minimum buffer capacity of 50 mM; adding to the solution oxygen scavenging substances such as ascorbic acid and anthocyanidins from berry juice powders to protect EGCG by competing for removal of dissolved oxygen; adding a trace element removal substance such as disodium EDTA; filling to the neck or shoulder in amber glass or PET containers, and blanketing the container headspace prior to capping.

### EXAMPLE 1

A liquid composition was prepared containing 7.3% short-chain fructooligosaccharides (scFOS) and 0.22% green tea extract, representing 1100 mg scFOS (10% overage) and 12 mg EGCG (20% overage) per 15 ml, respectively. In the example presented below, ascorbic acid and anthocyanidins from cranberry juice powder serve as oxygen scavengers. A person skilled in the art may easily select the best oxygen-scavenging substances for inclusion in the formulation without undue experimentation. The adjustment of pH to 4.80 is accomplished with 10 N NaOH solution.

A liquid composition was prepared by combining the following ingredients:

| Ingredients | Weight (%) |
|---|---|
| *Fructo-oligosaccharides | 7.3 |
| **Green Tea Extract | 0.22 |
| Carrageenan | 0.25 |
| Sodium citrate dihydrate | 0.86 |
| Citric acid anhydrous | 0.40 |
| Methylparaben | 0.12 |
| Sorbitol 70% | 4.00 |
| Xylitol | 5.00 |
| Disodium edetate | 0.10 |
| ***Cranberry juice powder | 0.35 |
| Cranberry flavor | 0.30 |
| Ascorbic acid | 0.20 |
| Purified water | 80.9 |

*NutraFlora ® scFOS ™ (GTC Nutrition Co)
**Greenselect ® (Indena)
***Cape Cod Biolab Corporation

### EXAMPLE 2:

This liquid composition differs from the previous example only in that scFOS and EGCG are increased to 23.17 and 0.35%, respectively and 1% elderberry juice powder (Cape Cod Biolab) replaces cranberry juice powder as additional oxygen scavenger in the formulation along with 0.20% ascorbic acid.

### EXAMPLE 3

This liquid composition differs from previous examples in that it contains 800 mg of scFOS and 200 mg of inulin (Raftiline GR, Orafti Food Ingredients, Netherlands, and 10 mg EGCG per 15 mL. Ascorbic acid (0.2%) and cranberry juice powder (0.35%) serve as oxygen scavengers.

### EXAMPLE 4

This liquid composition contains 3000 mg of scFOS, and 45 mg EGCG per 15 mL. Ascorbic acid (0.5%) and cranberry juice powder (1.50%) serve as f oxygen scavengers.

A liquid composition was prepared by combining the following ingredients:

| Ingredients | Weight (%) |
|---|---|
| *Fructo-oligosaccharides | 23.17 |
| **Green Tea Extract | 0.35 |
| ***EGCG > 98% purity | 0.22 |
| Carrageenan | 0.25 |
| Sodium citrate dihydrate | 0.86 |
| Citric acid anhydrous | 0.40 |
| Methylparaben | 0.12 |
| Fructose | 1.00 |
| Xylitol | 5.00 |
| Disodium edetate | 0.10 |
| ****Cranberry juice powder | 1.50 |
| Cranberry flavor | 0.30 |
| Ascorbic acid | 0.50 |
| Purified water | 66.23 |

*NutraFlora ® scFOS ™ (GTC Nutrition Co)
**Greenselect ® (Indena)
***Baralex, Montreal, Canada
****Cape Cod Biolab Corporation

### EXAMPLE 5:

Typically, the liquid composition useful for maintenance and restoration of colon functionality will be composed from about 3% to about 45% percent of short-chain fructooligosaccharides (scFOS) and from about 0.1 to about 0.8 percent of the green tea catechin, epigallocatechin gallate (EGCG). More preferably, about 1000 mg scFOS and 15 mg EGCG per teaspoonful (or 5 ml) will be administered in single or multiple doses for adults, and about 500 mg NDO and 5 mg EGCG per teaspoonful (or 5 ml) for children.

### EXAMPLE 6

Stability of scFOS in View of pH and Buffer Capacity

The inventors have found that the potency of short-chain fructooligosaccharides (scFOS) is maintained in sodium citrate—citric acid (50 mM) solutions buffered at pH 5.00 and 4.80 in HDPE bottles beyond 6 months of storage. The formulation also contained 0.2% ascorbic acid and 0.35% cranberry juice powder as flavoring agent. The scFOS used in this study consisted of a mixture of GF₂, GF₃ and GF₄ whereby two, three and four fructose units are linearly linked to a glucose molecule. The loss in potency was determined by a validated HPLC procedure consisting of a suitable HPLC system adjusted with a refractive index detector at 40° C., Supelcosil LC-NH₂ column and a mobile phase of acetonitrile and water. Similar solutions buffered at 4.60, 4.40 and 4.20 lost 9.5, 12 and 21 percent respectfully during the same six-month time interval. In similar liquid formulations with buffer capacities of 10 and 25 mM, a significant pH drift downward occurred in all samples during 6-months storage along with a corresponding enhanced degradation of FOS. Therefore the present inventors have found that the minimum pH requirement for optimal FOS stability in the liquid is of 4.80 and a buffer capacity of 50 mM.

Stability of EGCG and scFOS in view of pH

Next, the present inventors conducted a study to evaluate the effect of different pH values (4.20, 4.50 and 4.80) on the stability of EGCG in the same scFOS base formulation described hereinabove. The samples were filled into borosilicate glass tubes (13 mm×100 mm, Fisher Scientific, Montreal) and stored in an incubator at 50° C. for 28 days. Samples were removed at 7-day intervals for EGCG analyses by a validated HPLC procedure consisting of a suitable HPLC system equipped with a UV-Vis detector (wavelength 210-800 nm), a Nova-Pak C18 column and a mobile phase of acetonitrile and 0.1% aqueous acetic acid solution. At pH 4.20, only 1.085% loss/day was observed whereas similar losses of 1.414 and 1.413% were observed for the samples with pH 4.50 and 4.80, respectively. Therefore, the present inventors have found that a buffer system containing sodium citrate—citric acid with minimum 50 mM buffer capacity and a controlled pH value of 4.80 are minimum requirements for optimal EGCG and FOS stability.

EGCG Stability, Nitrogen Sparging and Container Headspace

Aqueous solutions of EGCG (2 mg/mL) from green tea extract were next utilized to examine the effects of nitrogen sparging and container headspace on EGCG stability when exposed to a temperature of 50° C. for 28 days. Samples were sparged with nitrogen for 30 and 60 minutes in separatory glass funnels, carefully filled into borosilicate glass tubes, the headspace blanketed with nitrogen, the top covered with two layers of saran wrap (to minimize evaporation), and the tube then closed with polypropylene screw caps. For the headspace study, solutions were filled to the shoulder or filled to 2 cm below the tube shoulder. Percent loss versus time plots for the nitrogen sparging experiment revealed no significant differences between control, 30-minute or the 60-minute sparging samples. However almost a twofold rate increase in EGCG loss was observed in the 2 cm headspace samples compared to the samples filled to the tube shoulder. Therefore, the inventors have found that preferred filling level requirement for EGCG-containing liquids is the shoulder or neck of the container.

EGCG Stability and Antioxidants

Next, similar aqueous solutions of EGCG (2 mg/ml) were spiked with the antioxidants Vitamin C, anthocyanidins, disodium ethylenediamine tetra acetic acid (EDTA), butylated hydroxytoluene (BHT), butylated hydroxyanisole (BHA) and sodium metabisulfite, and mixtures thereof and subjected to accelerated testing at 50° C. during 28 days as previously. For solubility and taste, the antioxidants Vitamin C, anthocyanidins, disodium ethylenediamine tetra acetic acid (EDTA), and mixtures thereof are preferred. The Excel-generated linear regression plots (least squares method) for percent EGCG concentrations versus time in days revealed the following degradation rate constants (k, %/day): 1.93 for pure EGCG (>98% purity Sigma, St. Louis); 1.35 for green tea catechins (GTC, >40% EGCG content, Greenselect, Indena, Italy); 1.02, 0.80 and 0.46 for GTC samples containing 0.10, 0.25 and 0.50% ascorbic acid, respectively; 0.90 for GTC sample containing 0.25% Cranberry Juice Powder (“CJP”, Cape Cod Biolab Corporation, Massachusetts)+0.1% disodium EDTA; 0.21 for GTC sample containing 0.20% ascorbic acid+0.35% CJP+0.1% disodium EDTA. The present inventors' results clearly demonstrate that EGCG integrity is enhanced in the presence of other oxygen or free radical scavengers, even with other catechins from GTC where the degradation rate is approximately ⅔ that of pure EGCG alone. In these formulations, CJP serves as both a flavor contributor and a source of anthocyanidins. Disodium EDTA is utilized to scavenge dissolved trace metals present in the CJP such as iron. Therefore, the present inventors have found that a preferred antioxidant is a combination of ascorbic acid, anthocyanidins from a berry juice powder and disodium EDTA.

EGCG Stability in View of Light and Oxygen Exclusion Storage

Next, the applicant investigated the stability of EGCG in the preferred antioxidant formulation stored in amber glass and amber polyethylene terephthalate (PET) bottles with black phenolic caps (Richards Packaging, Montreal, Canada) as well as the same borosilicate glass tubes and closures described previously. Previous assays of samples stored in high-density polyethylene (HDPE) bottles revealed extensive EGCG loss because of oxygen penetration. Samples were stored in incubators at 65° C. and removed at 2, 5, 7, 9 and 12-day intervals for EGCG analysis by HPLC. Percent EGCG rate losses per day were found to be 4.97, 4.20 and 4.65 for samples stored in tubes, glass and PET bottles respectfully. For light and oxygen exclusion purposes, the preferred container therefore may be either amber glass or amber PET bottles.

EGCG and FOS Stability and Shelf Life

Finally, accelerated shelf life testing (ASLT) was conducted on the formulation to ascertain the extent of degradation anticipated after 24 months storage at room temperature. The composition from Example I was filled to the necks of amber glass Winchester bottles (150 mL, Richards Packaging, Montreal, Canada), blanketed with nitrogen and capped with black phenolic polyseal closures. The containers were stored in incubators at 55, 65 and 75° C. during a 28-day time period and at pre-established daily time intervals triplicate containers were removed, stored under refrigeration and then analyzed for EGCG and scFOS according to HPLC procedures described above. Each percent concentration value for EGCG was the average of duplicate HPLC injections from each of three separate containers. scFOS values were the average of triplicate injections from single containers.

The degradation rate constants for EGCG degradation were obtained from Excel linear regression plots and were found to be 1.48, 3.25 and 10.76 percent per day for 550, 650 and 75° C., respectively. An Arrhenius plot of the logarithm of the rate constants (log k) versus the reciprocal of their absolute temperatures (1/K×1000) provided a straight line and demonstrated a good fit (r²=0.982). Extrapolation along this line permits one to predict the approximate k value for any selected temperature, for example at room temperature (22° C.) the k value is estimated to be 0.02% EGCG loss per day with a 24 month predicted loss of 14.4 percent. In the liquid composition described herein a 20% overage was included in the formulation so a proposed label claim of 10 mg/15 mL should be met after 2 years. It is noteworthy that the same formulation buffered at pH 5.0 displayed 10-12% faster degradation profiles than its pH 4.80 counterpart.

Degradation rate constants for scFOS degradation were also obtained from Excel linear regression plots and found to be 2.52, 6.45 and 17.39 percent per day for 550, 650 and 75° C., respectively. An Arrhenius plot of the logarithm of the rate constants (log k) versus the reciprocal of their absolute temperatures (1/K×1000) provided a straight line and demonstrated an extremely good fit (R²=0.999). The estimated k value at 22° C. is 0.0316% scFOS loss per day with a 24 month predicted loss of 22.8 percent. In the liquid composition described in Example I only a 10% overage was included so a 20-25% overage may be required in order to meet a proposed label claim of 1000 mg scFOS/15 mL after 2 years.

Actual percent losses at 3 and 6 months versus predicted values to date appear in the following table and demonstrate that a 24-month expiry dating is possible with this formulation.

| | |
|---|---|
| EGCG Loss (%) | FOS Loss (%) |

| Time (mo) | Predicted | Actual | Predicted | Actual |
|---|---|---|---|---|
| 3 | 1.80 | 2.49 | 2.85 | 1.95 |
| 6 | 3.59 | 3.39 | 5.69 | 3.16 |
| 24 | 14.36 | | 22.77 | |

ASLT testing was also conducted on the composition from Example 2 and the actual percent losses at 3 and 6 months versus predicted values appear in the following table and again demonstrates the potential for a 24-month expiry dating.

| | |
|---|---|
| EGCG Loss (%) | FOS Loss (%) |

| Time (mo) | Predicted | Actual | Predicted | Actual |
|---|---|---|---|---|
| 3 | 2.26 | 2.86 | 3.58 | 0.43 |
| 6 | 4.52 | 4.60 | 7.17 | 3.92 |
| 24 | 18.09 | | 28.66 | |

While several embodiments of the invention have been described, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention as described herein.

What is claimed is:

1. A liquid composition for providing restored or maintained colon functionality comprising an effective amount of a non-digestible oligosaccharide, at least one green tea catechin and a buffering agent mixture, said liquid composition being in a pH range of from about 4.7 to about 5.0.

2. A liquid composition for preventing cancer comprising an effective amount of a non-digestible oligosaccharide, at least one green tea catechin and a buffering agent mixture, said liquid composition being in a pH range of from about 4.7 to about 5.0.

3. The liquid composition of claim 1 or 2, characterised in that the non-digestible oligosaccharide is chosen from the group consisting of xylo-oligosaccharides, soyoligosaccharides, fructo-oligosaccharides, trans-galacto-oligosaccharides, palatinose condensates, isomalto-oligosaccharides, inulin, pyrodextrin, and mixtures thereof.

4. The liquid composition of claim 1 or 2, characterised in that the non-digestible oligosaccharide is chosen from the group consisting of oligofructose, short chain fructo-oligosaccharides, and mixtures thereof.

5. The liquid composition of claim 3, characterised in that the fructo-oligosaccharide is chosen from the group consisting of short-chain fructo-oligosaccharides, and mixtures thereof.

6. The liquid composition of claim 4 or 5, characterised in that the short-chain fructo-oligosaccharide has a maximum degree of polymerisation (DP) of 4.

7. The liquid composition of any one of claims 1 to 6, characterised in that the non-digestible oligosaccharide is at a concentration of about 3% to about 45% by weight.

8. The liquid composition of any one of claims 1 to 7, characterised in that the at least one green tea catechin is selected from the group consisting of epicatechin (EC), epigallocatechin (EGC), epicatechin gallate (ECG) and epigallocatechin gallate (EGCG), and mixtures thereof.

9. The liquid composition of claims 8, characterised in that the at least one green tea catechin is epigallocatechin gallate (EGCG).

10. The liquid composition of claim 9, characterised in that the epigallocatechin gallate (EGCG) is at a concentration of from about 0.1 to about 0.8% by weight.

11. The liquid composition of any one of claims 8 to 10, characterised in that the epigallocatechin gallate (EGCG) is derived from a decaffeinated green tea plant extract having an EGCG content of from about 25 to about 99% by weight.

12. The liquid composition of any one of claims 8 to 11, characterised in that it further comprises an antioxidant chosen from the group consisting of water-soluble or water-dispersible oxygen scavenging agents, and mixtures thereof.

13. The liquid composition of claim 12, characterised in that the oxygen or free radical scavenging agent is selected from the group consisting of butylated hydroxytoluene (BHT), butylated hydroxyanisole (BHA), tocopherols, ascorbic acid, ascorbic acid salts, anthocyandins from fruit juice powder, anthocyandins from fruit juice concentrate, anthocyandins from vegetable juice powder, anthocyandins from vegetable juice concentrate, and mixtures thereof.

14. The liquid composition of claim 13, characterised in that the oxygen or free radical scavenging agent consists of ascorbic acid and anthocyandins from berry juice powders.

15. The liquid composition of any one of claims 12 to 14, characterised in that the antioxidant is at a concentration of from about 0.1% to about 5% by weight.

16. The liquid composition of any one of claims 12 to 15, characterised in that it further comprises a trace metal ion scavenger.

17. The liquid composition of 16, characterised in that the trace metal ion scavenger is selected from group consisting of ethylene diamine tetracetic acid (EDTA) and salts thereof, and mixtures thereof.

18. The liquid composition of claim 16 or 17, characterised in that the trace metal ion scavenger is at a concentration of from about 0.05% to about 0.25% by weight.

19. The liquid composition of any one of claims 16 to 18, characterised in that the buffering agent mixture selected from group consisting of citrates, phosphates, acetates, ascorbates, and mixtures thereof.

20. The liquid composition of claim 19, characterised in that the buffering mixture comprises sodium citrate and citric acid.

21. The liquid composition of claim 19 or 20, characterised in that the buffering agent mixture is at a concentration of from about 0.1% to about 2% by weight.

22. A method of restoring and maintaining colon functionality in a human being, said method comprising administering to said human being an effective amount of a liquid composition of any one of claims 1 to 21 to restore and maintain colon functionality in said human being.

23. A method of helping prevent cancer in a human being, said method comprising administering to said human being an effective amount of a liquid composition of any one of claims 1 to 21 to help prevent cancer in said human being.

24. Use of an effective amount of a liquid composition of any one of claims 1 to 21 to restore and maintain colon functionality in a human being.

25. Use of an effective amount of a liquid composition of any one of claims 1 to 21 to help prevent cancer in a human being.

26. Use of an effective amount of a liquid composition of any one of claims 1 to 21 for the manufacture of a medicament useful for restoring or maintaining colon functionality in a human being.

27. Use of an effective amount of a liquid composition of any one of claims 1 to 21 for the manufacture of a medicament useful for in helping to prevent cancer in a human being.

28. A method for increasing the stability of short-chain fructo-oligosaccharides and epigallocatechin gallate (EGCG) in liquid compositions comprising the steps of:
a) buffering the said liquid with sodium citrate and citric acid in a pH range of from about 4.70 to about 5.0 and a minimum buffer capacity of 50 mM;
b) adding to the said liquid an oxygen scavenging agent to compete for removal of dissolved oxygen; and
c) adding a trace metal ion scavenger to remove trace metal ions.

29. The method according to claim 28, characterised in that it further comprises the steps of:
d) filling to neck or shoulder level in amber glass or PET container; and
e) blanketing container headspace prior to capping said container.

30. The method according to claim 28 or 29, characterised in that the liquid composition is the liquid composition of any one of claims 3 to 21.
